(19) United States
(12) Patent Application Publication
(10) Pub. No.: US 2009/0270979 A1
(43) Pub. Date: Oct. 29, 2009

(54) BIODEGRADABLE METALLIC STENT

(75) Inventor: Nina Adden, Nuernberg (DE)

Correspondence Address: BRYAN CAVE POWELL GOLDSTEIN, ONE ATLANTIC CENTER FOURTEENTH FLOOR, 1201 WEST PEACHTREE STREET NW, ATLANTA, GA, 30309-3488, US

(73) Assignee: BIOTRONIK VI PATENT AG, Baar (CH)

(21) Appl. No.: 12/429,898

(22) Filed: Apr. 24, 2009

(30) Foreign Application Priority Data
Apr. 24, 2008 (DE) 10 2008 020 415.3

(51) Int. Cl. A61F 2/06

(52) U.S. Cl. 623/1.46

(57) ABSTRACT

A biodegradable metal stent having delayed degradation after implantation, the stent comprising a stent material coated with at least one wax material, Also provided is a method for manufacturing and use of such a stent.

### PRIORITY CLAIM

This patent application claims priority to German Patent Application No. 10 2008 020 415.3, filed Apr. 24, 2008, the disclosure of which is incorporated herein by reference in its entirety.

### FIELD

The present disclosure relates to a biodegradable metal stent, a method for manufacturing a stent and a method for delaying the degradation of an inventive biodegradable metal stent after implantation in a human or animal body.

### BACKGROUND

Stents in general are endovascular (peripheral or coronary) prostheses and/or implants that are used for treatment of stenoses, for example. Stents are also known for treatment of aneurysms.

Stents essentially have a supporting structure suitable for supporting the wall of a vessel in such a manner as to widen the vessel and/or to bridge an aneurysm. Stents are, therefore, in a compressed state when inserted into the vessel and are then widened at the treatment site and are pressed against the vascular wall. This dilatation may be accomplished with the help of a balloon catheter, for example. Alternatively, self-expanding stents are also known. These are constructed of a super-elastic metal such as Nitinol, for example.

Stents are currently divided into two basic types, permanent stents and biodegradable stents. Permanent stents are designed to remain in a blood vessel for an indefinite length of time. Biodegradable stents, however, are degraded in the vessel over a predetermined period of time. Biodegradable stents are preferably degraded only when the traumatized tissue of the vessel has healed and the stent no longer needs to remain in the vascular lumen.

For example, known biodegradable stent materials include biodegradable metal alloys, polymers or composite materials which have a sufficient structural load-bearing capacity to be able to support the vascular lumen for a predetermined period of time.

However, it has been found that due to the introduction and retention of the stents in the vascular systems, late complications may often occur, e.g., in-stent restenoses, vasculitis and thromboses.

For this reason, stents have been further developed to also comprise, in addition to their supporting structure, one or more active ingredients which are released directly and topically to the human or animal body during and/or after implantation to thereby reduce the incidence of complications.

An active ingredient is usually applied to the surface of the stent base body by means of a polymer carrier, such that the active ingredient is released from the polymer matrix by diffusion and/or erosion processes after implantation.

If a nondegradable polymer is used as the polymer carrier, the nondegradable polymer may increase the risk of thrombosis because the polymer remains as a foreign body in the patient's body.

When degradable polymers (e.g., polyesters) are used as the polymer carriers, the polymer degradation products formed during degradation may cause vasculitis or even tissue necrosis. On the other hand, the resulting polymer degradation products can also act directly on a biodegradable metal stent base body. The pH in the immediate vicinity of the biodegradable metal stent drops because the polymer degradation products are mainly acidic (for example, lactic acid, glycolic acid, and the like). Due to the pH shift into the acid range, the rate of decomposition of a biodegradable metal stent base body can be influenced in a negative sense.

As an alternative to an active ingredient coating by means of a polymer carrier, oil and fat coatings containing an active ingredient have been investigated in medical products (see, for example, International Patent Publication Nos. WO 03/039612, WO 2005/053767 and WO 2006/036983). It has been found that although these carrier materials do not cause any significant negative physiological reactions in the human or animal body after implantation, such oil or fat coatings in some cases do not adhere adequately to the stent surface due to their mechanical properties.

To eliminate this circumstance, oils, which are usually liquid, have been either partially hydrogenated (International Patent Publication No. WO 2005/053767) or partially crosslinked (International Patent Publication No. WO 2006/036983) to achieve a sufficiently solid consistency and thereby allow sufficient adhesion to the stent.

But even in the case when oil and fat coatings adhere adequately to the stent surface, such coatings usually still do not have adequate mechanical properties in practice. For example, if a stent is coated with an oil and fat coating before crimping on a catheter, then the coating will show damage after crimping.

Furthermore, an oil and fat coating usually has a low scratch resistance so the risk of damage to the coating, e.g., due to friction of the guide catheter on the mural coated side of the stent (i.e., in a typically cylindrical stent, the outside cylindrical surface, i.e., the surface facing the tissue and not the surface facing the vascular lumen) is high on penetration into the body. Such damage may cause the stent material to come in contact with physiological fluids so that, in the case of a biodegradable stent, the degradation begins at this point in time. If the oil and fat coating additionally contains one or more active ingredients, then the active ingredient concentration to be released may no longer be sufficient, due to this damage to the coating, to induce the desired physiological effects.

The present disclosure provides a biodegradable metal stent which is simple to manufacture, which has a coating which does not cause any unwanted physiological reactions, which has sufficient mechanical properties, i.e., the stent can be coated before crimping and the coating remains functional even after crimping and/or the coating has a sufficient scratch resistance so that it is not damaged, in particular, by a guide catheter on insertion into a human or animal body, and/or whereby degradation begins with a time lag in comparison with polymer-coated biodegradable metal stents.

### SUMMARY

The present disclosure describes several exemplary embodiments of the present invention.

One aspect of the present disclosure provides a biodegradable metal stent, comprising a stent material whereby the stent surface is coated with a wax layer.

Another aspect of the present disclosure provides a method for manufacturing a coated biodegradable metal stent, comprising a) providing at least one wax; b) preparing either a melt or a solution with at least one solid of the wax from step a); c) providing a biodegradable metal stent; and, d) coating the surface of the stent from step c) with either the melt or the solution of the wax from step b).

A further aspect of the present disclosure provides a method for delaying the degradation of a biodegradable stent after implantation in a human or animal body, comprising coating a biodegradable metal stent with at least one wax material and implanting the coated stent into a human or animal body.

Exemplary embodiments of the present disclosure are described in the claims and in the following detailed description and, if appropriate, can be combined with one another.

### DETAILED DESCRIPTION

The present invention is a biodegradable metal stent coated according to the present disclosure which addresses one or more of the problems described herein above through the choice one or more waxes as the coating material.

The wax coating according to the present disclosure has a higher scratch resistance in comparison with a corresponding oil or fat coating so that after using a guide catheter for introduction into the human or animal body, for example, the coating is not damaged in such a way that physiological fluids would come in contact with the stent material so that degradation of the stent would begin then. In other words, the wax layer of the present disclosure remains functional after being introduced into the human or animal body by means of the guide catheter.

In addition, the inventive wax layer is more suitable for coating the surface of a stent before crimping in comparison with an oil and fat coating because the wax coating remains functional even after crimping.

According to one exemplary embodiment, for the case when the wax layer contains one or more active ingredients, there is a reduced risk that the coating will not have an effective concentration of active ingredient and/or will not effectively release the active ingredient after implantation.

Since the wax coating remains functional even after implantation in the human or animal body, the wax coating is hydrophobic and does not swell on contact with physiological fluids; in particular, the degradation of a biodegradable metal stent is delayed in comparison with a biodegradable metal stent having a polymer coating because the polymer coating usually swells on contact with physiological fluids. One advantage of delayed degradation is that the stent base body does not lose its integrity for a longer period of time and, therefore, support of the vessel can be guaranteed for a longer period of time.

Since the wax layer also does not supply any acidic degradation products in comparison with a degradable polymer layer, there is no additional negative influence on the rate of decomposition of the biodegradable metal stent base body due to the wax coating.

Furthermore, in the implanted state, the wax coating also does not have any adverse physiological effects. This further reduces the risk of a late complication.

Moreover, the biodegradable metal stent of the present disclosure can be manufactured easily with a wax layer because conventional waxes that are suitable for pharmaceutical purposes are obtainable commercially on the one hand and on the other hand need only be dissolved in a suitable solvent in order to allow coating of the stent. Hydrogenation steps and/or crosslinking steps such as those usually required with oil and fat coatings are not necessary to produce the wax coating of the present disclosure.

For purposes of the present disclosure, the terms “biodegradable metal stent” and “biodegradable stent” mean that the base body of the metal stent degrades, i.e., decomposes in a physiological environment, in particular, in the vascular system of a human or animal body, so that the stent loses its integrity. The biodegradable metal stent base body preferably degrades only when the function of the stent is no longer physiologically appropriate and/or necessary. With biodegradable metal stents, this means that the stent is preferably degraded or loses its integrity only when the traumatized tissue of the vessel has healed and thus the stent no longer needs to remain in the vascular lumen.

According to the present disclosure, the biodegradable metal stent comprises a metallic material which is a biocorrodible alloy such that the main component of the alloy is selected from the group consisting of magnesium, iron, zinc and tungsten. A magnesium alloy, in particular, is preferred for a degradable metallic material.

The alloy, comprising, in particular, magnesium, iron, zinc and/or tungsten, is to be selected in its composition so that the alloy is biocorrodible. For purposes of the present disclosure, “biocorrodible” refers to alloys in which a degradation takes place in a physiological environment, ultimately resulting in the entire stent or the part of the stent formed by the material losing its mechanical integrity. For purposes of the present disclosure, the term “alloy” means a metallic structure whose main component is selected from the group consisting of magnesium, iron, zinc or tungsten. The main component is the alloy component present in the alloy in the largest amount by weight. The amount of the main component is preferably more than 50 wt %, more preferably more than 70 wt %. A magnesium alloy is preferred.

If the material is a magnesium alloy, then the material preferably contains yttrium and other rare earth metals because such an alloy is excellent with regard to its physicochemical properties and its high biocompatibility, in particular, its degradation products.

Magnesium alloys of the WE series, in particular, WE43, and magnesium alloys of the following composition are especially preferred: 5.5-9.9 wt % rare earth metals, including 0.0-5.5 wt % yttrium and <1 wt % remainder, which may include zirconium and/or silicon, and magnesium accounts for the remaining alloy up to 100 wt %. These magnesium alloys have already confirmed their special suitability in experimental studies and preliminary clinical trials, i.e., the magnesium alloys have shown a high biocompatibility, favorable processing properties, good mechanical characteristics and adequate corrosion behavior for the intended purpose. For purposes of the present disclosure, the collective term “rare earth metals” includes sandium (21), yttrium (39), lanthanum (57) and the 14 elements following lanthanum (57), namely cerium (58), neodymium (60), promethium (61), samarium (62), europium (63), gadolinium (64), terbium (65), dysprosium (66), holmium (67), erbium (68), thulium (69), ytterbium (70) and lutetium (71).

For purposes of the present disclosure, all the conventional stent geometries may be used as the biodegradable metal stents. Especially preferred stent geometries are described in U.S. Pat. No. 6,896,695; U.S. Patent Publication No. 2006/241742; U.S. Pat. No. 5,968,083 (Tenax); European Patent Application No. 1 430 854 (helix design); U.S. Pat. No. 6,197,047; and European Patent Application No. 0 884 985, for example.

For purposes of the present disclosure, the terms “wax layer” or “wax coating” mean that the surface of the biodegradable metal stent is completely or partially coated with a layer comprising one or more waxes.

The surface of the metal stent is coated with the wax layer on the luminal side, i.e., in the case of a typical cylindrical stent, the surface facing the central axis of the cylinder as well as the surface on the mural side, i.e., the outer cylindrical surface in the case of a typical cylindrical stent. In an especially preferred exemplary embodiment, the stent surface is coated with the wax layer only on the mural side so that degradation of the stent begins from the luminal side but not from the mural side.

For purposes of the present disclosure, the term “wax” is a collective term for a number of natural substances (of plant, animal or mineral origin) or synthetically produced substances which are generally extensible at room temperature, melt above 45° C. without decomposing, have a low viscosity in the molten state and are insoluble and hydrophobic in water. The waxes are usually mixtures of esters of higher linear fatty acids (C₁₈ to C₃₄ or more) with higher alcohols (usually monovalent alcohols of the same length). For example, palmitates, palmitoleates, hydroxypalmitates and oleate esters of long alcohols (C₃₀ to C₃₂) are the main components of beeswax. Small amounts of free acids and alcohols of a similar length may also be present.

Suitable waxes are usually selected from the group consisting of natural or synthetic waxes which are suitable for use in pharmaceuticals, in particular, and here, specifically, for implantation in a human or animal body, i.e., pharmaceutical grade according to Ph. Eur. 5 (European Pharmacopeia 5).

The wax layer preferably comprises or consists of one or more natural waxes. The natural waxes are especially preferably selected from the group consisting of: animal waxes, including white wax (cera alba), beeswax (cera flava) and lanolin (wool fat and/or adeps lanae); and/or vegetable waxes including simmondsia wax (jojoba oil), carnauba wax (Brazil wax) and candelilla wax (kanutilla wax).

Beeswax is preferred, in particular, because of its extensibility, whereas carnauba wax is preferred, in particular, because of its scratch resistance which allows coating of the stent before crimping. In addition, candelilla wax is harder than beeswax but softer than carnauba wax.

In another exemplary embodiment, the desired mechanical properties can be better adapted to the necessary requirements by mixing two or more waxes. Preferred weight ratios of a mixture of two of the waxes are from 1:99 to 99:1, more preferably 90:10 to 10:90, especially preferably 80:20 to 20:80 and most especially preferably 70:30 to 30:70. A mixture of beeswax and carnauba wax is most especially preferably used for this.

In another exemplary embodiment, by adding free fatty acids, the desired mechanical properties can be better adapted to the required needs. Suitable fatty acids may preferably be selected from the group consisting of saturated fatty acids, e.g., butyric acid, valeric acid, caproic acid, enanic acid, caprylic acid, pelargonic acid, capric acid, lauric acid, myristic acid, palmitic acid, margaric acid, stearic acid, arachic acid, behenic acid; monounsaturated fatty acids, e.g., palmitoleic acid, oleic acid, elaidic acid, vaccenic acid, icosenic acid, cetoleic acid; or polyunsaturated fatty acids, e.g., linoleic acid, arachidonic acid, timnodonic acid, clupandonic acid, cervonic acid. Saturated fatty acids such as caprylic acid, pelargonic acid, capric acid, lauric acid, myristic acid and palmitic acid are especially preferred.

In another exemplary embodiment, the stent base body may be pretreated with a fatty acid solution as an adhesion promoter to achieve better adhesion of the wax to the stent. Suitable fatty acid solutions as adhesion promoters preferably comprise saturated fatty acids, e.g., butyric acid, valeric acid, caproic acid, enanic acid, caprylic acid, pelargonic acid, capric acid, lauric acid, myristic acid, palmitic acid, margaric acid, stearic acid, arachic acid or behenic acid. Laurie acid may especially preferably be used for this.

In another exemplary embodiment, the wax layer additionally comprises one or more active ingredients.

For purposes of the present disclosure, an active ingredient is a substance or a compound which causes a biological reaction in the human or animal body. In this sense, an active ingredient may also be considered to be synonymous with drug or pharmaceutical. For purposes of the present disclosure, the stent is coated with one or more active ingredients in a concentration sufficient to induce the desired physiological reactions.

Active ingredients to be used according to the present disclosure are preferably selected from the group consisting of anti-inflammatories, preferably dexamethasone, methylprednisolone and diclofenac; cytostatics, preferably paclitaxel, colchicine, actinomycin D and methotrexate; immunosuppressants, preferably limus drugs, more preferably sirolimus (rapamycin), zotarolimus (Abt-578), tacrolimus (FK-506), everolimus, biolimus, in particular, biolimus A9 and pimecrolimus, cyclosporin A and mycophenolic acid; platelet aggregation inhibitors, preferably abciximab and iloprost; statins, preferably simvastatin, mevastatin, atorvastatin, lovastatin, pitavastatin and fluvastatin; and estrogens, preferably 17β-estradiol, daizein and genistein; lipid regulators, preferably fibrates; immunosuppressants; vasodilators, preferably satans; calcium channel blockers; calcineurin inhibitors, preferably tacrolimus; anti-inflammatory drugs, preferably imidazoles; antiallergics; oligonucleotides, preferably decoy oligodeoxynucleotide (dODN); endothelial cell producing agents, preferably fibrin; steroids; proteins/peptides; proliferation inhibitors; analgesics and anti-rheumatic drugs.

According to the present disclosure, paclitaxel and limus compounds, more preferably sirolimus (rapamycin), zotarolimus (Abt-578), tacrolimus (FK-506), everolimus, biolimus, in particular, biolimus A9 and pimecrolimus, most especially preferably rapamycin (sirolimus), are especially preferred for use as the additional active ingredients.

In an especially preferred exemplary embodiment, the stent is completely or partially coated with the wax layer containing the active ingredient, preferably completely coated only on the mural surface of the stent. This preferred coating containing an active ingredient has the advantage that first, the active ingredients are released directly at the target site of the tissue and there are no relevant systemic adverse effects. Secondly, a corresponding coating containing an active ingredient has a reduced risk of thrombosis in comparison with a coating on both the luminal and mural surfaces because it has been observed that when both the mural and luminal surfaces of a stent have a coating containing an active ingredient, endothilialization of a stent coated in this way (growth of vascular cells through the stent) is delayed or prevented and, therefore, the risk of thrombosis is increased.

In another preferred exemplary embodiment, the stent may also have another wax layer without any active ingredient on the luminal surface in addition to having a mural coating containing an active ingredient. Such a coating has the advantage that, if desired, degradation of the stent is further delayed.

In another preferred exemplary embodiment, a stent coated according to the present disclosure may additionally have one or more other coatings as so-called topcoats which are completely or partially coated with the wax layer on the surface. Such topcoats may be free of active ingredient or may contain one or more active ingredients.

In a preferred exemplary embodiment, a stent has a wax layer free of active ingredient and has a topcoat containing one or more active ingredients. The wax layer assumes the function of degradation control and additionally protects the active ingredient contained in the topcoat from degradation products of the stent base body.

In the case when a stent is coated with a wax layer containing an active ingredient, a preferred active-ingredient-free topcoat assumes the function that the abrasion of the active ingredient coating of the wax layer is reduced in addition to the material properties of the wax layer which are already present.

The same materials and/or preferred exemplary embodiments of the wax layer may be used as the topcoat. Alternatively or cumulatively, one, two or more conventional polymers may be included, selected from the group consisting of:
nondegradable polymers: polyethylene; polyvinyl chloride; polyacrylates; preferably polyethyl and polymethyl acrylates, polymethyl methacrylate, polymethyl-co-ethyl acrylate and ethylene/ethyl acrylate; polytetrafluoroethylene, preferably ethylene/chlorotrifluoroethylene copolymers, ethylene/tretrafluoroethylene copolymers; polyamides, preferably polyamideimide, PA-11, PA-12, PA-46, PA-66; polyetherimide; polyethersulfone; poly(iso)butylene; polyvinyl chloride; polyvinyl fluoride; polyvinyl alcohol; polyurethane; polybutylene terephthalate; silicones; polyphosphazenes; polymer foams, preferably polymer foams of carbonates, styrenes; copolymers and/or blends of the polymer classes listed above, polymers of the thermoplastics class, and
degradable polymers: polydioxanone; polyglycolide; polycaprolactone; polylactides, preferably poly-L-lactide, poly-D,L-lactide, and copolymers as well as blends thereof, preferably poly(L-lactide-co-glycolide), poly(D,L-lactide-co-glycolide), poly(L-lactide-co-D,L-lactide), poly(L-lactide-co-trimethylene carbonate); triblock copolymers; polysaccharides, preferably chitosan, levan, hyaluronic acid, heparin, dextran, cellulose; polyhydroxy valerate; polyphosphazene; polyethylene oxide; poly-phosphorylcholine; fibrin; albumin; polyhydroxybutyric acid, preferably atactic, isotactic and/or syndiotactic polyhydroxybutyric acid and blends thereof.

Materials for a topcoat selected from the group consisting of waxes or degradable polymers are especially preferred. Carnauba wax is most especially preferred for use as the topcoat.

The preferred exemplary embodiments of the stent may be combined with one another in all conceivable variants but also with the other preferred exemplary embodiments disclosed hereinabove.

According to a second embodiment of the present disclosure a manufacturing process is provided for formation of wax-coated stents, preferably inventive stents.

The coating of the surface of the stent is applied by conventional methods. The wax or wax mixture is usually prepared by dissolving two, three or more waxes in a suitable solvent.

Suitable solvents are preferably selected from the group consisting of: cyclohexane, chloroform, acetone, petroleum ether, ethyl acetate, tetrahydrofuran (THF) and dichloromethane.

Beeswax is preferably dissolved in cyclohexane, chloroform and THF. Carnauba wax is preferably dissolved in chloroform and 40° C. also in dichloromethane and cyclohexane. Candelilla wax is preferably dissolved in acetone or petroleum ether.

The wax solution obtained in this way is applied to a stent and/or a crimped stent on a catheter once, twice or several times by using the following conventional methods: dipping methods (dip coating), spray coating by means of single-substance nozzles and/or multi-substance nozzles, rotary atomization and pressurized nozzles, paintbrushes, printing, and the like.

If necessary, a conventional drying step or other conventional physical or chemical aftertreatment steps, e.g., a vacuum or plasma treatment may follow after one or more coating steps before the stent is treated further.

If the wax layer contains one or more active ingredients, the active ingredients are dissolved or suspended in the solution in a sufficient concentration before the stent or the crimped stent on the catheter is coated with the solution/suspension by means of the methods disclosed hereinabove. The concentration of the active ingredients on the coated stent is usually such that the each active ingredient induces the desired physiological reaction either alone or in combination.

For the case when primarily only the mural surface of an stent is to be coated with a wax layer containing the active ingredient, this may preferably be accomplished by attaching the stent base body to a cylinder, cannula or mandrel, for example, and/or crimping it onto a catheter in the processes mentioned hereinabove. Alternatively, the abluminal coating may be performed with other active ingredients by roller application or by selective application by painting or by filling cavities.

The coating methods for the wax layer may also be applied to the topcoat.

A third exemplary embodiment of the present disclosure relates to the use of a wax coating on the surface of a biodegradable metal stent to delay the degradation of the stent after implantation in a human or animal body.

A fourth exemplary embodiment of the present disclosure relates to a method for delaying the degradation of a biodegradable metal stent after implantation in a human or animal body, wherein the stent is coated with a wax layer.

### EXAMPLES

The present disclosure is described hereinbelow by exemplary embodiments, although the exemplary embodiments do not restrict the scope of protection of the subject matter of the present disclosure.

### Exemplary Embodiment 1

### Wax Coating on a Pre-Crimped Stent

A stent of the biocorrodible magnesium alloy WE43 (97 wt % magnesium, 4 wt % yttrium, 3 wt % rare earth metals, not including yttrium) is crimped onto a catheter, cleaned to remove dust and residues and then coated as follows: Beeswax (Gustav Hees) is melted at 60° C. The tip of the catheter is immersed in the melt and extracted slowly. After a drawing time of 1 minute, the protector is placed on the catheter again.

### Exemplary Embodiment 2

### Wax Coating on an Uncrimped Stent

A stent of the biocorrodible magnesium alloy WE43 (97 wt % magnesium, 4 wt % yttrium, 3 wt % rare earth metals, not including yttrium) is cleaned to remove dust and residues and pretreated for 16 hours in a solution of lauric acid in chloroform.

A wax mixture is prepared as follows: 3 wt % of a mixture of 80 wt % beeswax (Gustav Hees) and 20 wt % Carnauba wax (Gustav Hees) is dissolved in chloroform, where the amounts by weight are based on the resulting mixture.

The stent is attached to a hook. Then the stent is immersed in the wax mixture under constant ambient conditions (room temperature (RT); 42% atmospheric humidity) with the help of a dipping system (Specialty Coating Systems) and is pulled out of the wax mixture at the rate of 1 mm per minute.

The stent is dried for 5 minutes at RT. Additional dipping passes are also possible.

The completely coated stent is dried for 16 hours at 40° C. in a vacuum oven (Vakucell; MMM).

### Exemplary Embodiment 3

### Wax Coating Containing an Active Ingredient on an Uncrimped Stent

A PRO-KINETIC® stent (BIOTRONIK) is cleaned in chloroform.

A wax mixture is prepared as follows: 3 wt % of a mixture of 70 wt % beeswax (Gustav Hees) and 30 wt % carnauba wax (Gustav Hees) are dissolved in chloroform (weight amounts based on the resulting mixture); 30 wt % rapamycin, based on the wax content in the mixture, is added and the mixture is stirred.

The stent is clamped in a suitable stent coating apparatus (DES coater, in-house development by Biotronik), by clamping half of the stent in the apparatus so that the other half of the stent can be coated. With the help of the air brush system (EFD or Spraying System), the rotating stent is coated under constant ambient conditions (room temperature; 42% atmospheric humidity) on the half of the stent which is not clamped in the apparatus.

After achieving the intended layer weight of approx. 300 μg, the stent is dried at RT for 5 minutes before coating the uncoated end similarly after unclamping the stent from the coating apparatus, turning the stent around and clamping the coated half of the stent again.

The completely coated stent is dried for 16 hours at 60° C. in a vacuum oven (Vakucell™; MMM).

All patents, patent applications and publications referred to herein are incorporated by reference in their entirety.

What is claimed is:

1. A biodegradable metal stent, comprising a stent material whereby the stent surface is coated with a wax layer.

2. The stent of claim 1, wherein the wax layer comprises at least one natural wax.

3. The stent of claim 2, wherein the natural wax is selected from the group consisting of white wax, beeswax, lanolin, simmondsia wax, carnauba wax and candelilla wax.

4. The stent of claim 1, wherein the wax layer comprises at least one active ingredient.

5. The stent of claim 4, wherein the wax layer containing the active ingredient is applied to the mural surface of the stent by coating.

6. A method for manufacturing a coated biodegradable metal stent, comprising:
a) providing at least one wax;
b) preparing either a melt or a solution with at least one solid of the wax from step a);
c) providing a biodegradable metal stent; and
d) coating the surface of the stent from step c) with either the melt or the solution of the wax from step b).

7. A method for delaying the degradation of a biodegradable stent after implantation in a human or animal body, comprising coating a biodegradable metal stent with at least one wax material and implanting the coated stent into a human or animal body.
